(19) United States
(12) Patent Application Publication
(10) Pub. No.: US 2022/0401024 A1
(43) Pub. Date: Dec. 22, 2022

(54) SYSTEMS AND METHODS FOR MEDICAL DEVICE ANCHORING

(71) Applicant: University of Maryland, College Park, College Park, MD (US)

(72) Inventors: Ashley CHAPIN, Washington, DC (US); Luke A. BEARDSLEE, Atlanta, GA (US); Reza GHODSSI, Potomac, MD (US); Joshua Aaron Levy, College Park, MD (US)

(21) Appl. No.: 17/839,451

(22) Filed: Jun. 13, 2022

Related U.S. Application Data
(60) Provisional application No. 63/209,961, filed on Jun. 11, 2021.

(51) Int. Cl. A61B 5/00; A61B 5/145; A61B 5/0537; A61B 5/0538; A61M 37/00; A61K 9/20

(52) U.S. Cl. CPC A61B 5/685; A61B 5/14546; A61B 5/0537; A61B 5/0538; A61M 37/0015; A61K 9/20; A61B 2560/063; A61B 2562/0215; A61M 2037/003; A61M 2037/0038; A61M 2037/0023; A61M 2037/0053

(57) ABSTRACT

This disclosure sets forth various systems and methods for deploying anchored medical devices within a human or animal. The medical devices may deliver payloads, such as various sensors, electrodes, transmitters, cameras, electrical or other interventional devices, drugs or therapeutics. The devices may have one or more anchors, which attach the device to an anatomy of interest. This allows for methods and processes to be performed over periods of time, such as extended delivery of a therapy or real time sensing of characteristics inside a body, which the device remains within a given location.

### CROSS-REFERENCE TO RELATED APPLICATIONS

This application claims priority to U.S. Provisional Patent Application No. 63/209,961, filed Jun. 11, 2021, the entire content of which is incorporated herein by reference in its entirety.

### BACKGROUND

There are a number of conditions existing within the human (or animal) body, for which current standard of care would involve attempting to sense a condition or deliver a therapeutic to a particular, localized anatomy of interest from within the body. However, given the limitations of existing technologies for targeting specific anatomical locations within a patient, it is difficult to perform these procedures over a period of time or consistently at the same precise location. For example, it is difficult to obtain real-time detection of biomarkers released from and/or to provide direct application of therapeutics at certain locations within the body of a patient, for example, at the basolateral side of the GI epithelium, over a continuous or lengthy period of time. Systems exist for monitoring some biomarkers and/or for providing some therapeutics, however, these systems typically must be handled and inserted into a patient manually. This type of insertion and positioning involves a variety of limitations. For example, it may be difficult to maintain stable positioning of a device in the gut of a patient, because the gut is influenced by muscles which move constantly, and also which are typically only accessible through endoscopic means, and the duration of measurement is limited to the duration of the medical procedure. Similarly, targeted and/or timed delivery of a therapeutic can be difficult for certain anatomies, given their location. For example, human and animal GI tracts are not naturally amenable to sustained location of a therapeutic delivery agent, given their movements.

Therefore, it would be desirable if a medical device/system could provide for sustained, in vivo delivery of various payloads, such as sensing components and/or therapeutics.

### SUMMARY

In accordance with some embodiments of the disclosed subject matter, provided is an in vivo delivery device for attaching to tissue within the body of a patient, the in vivo delivery device including a housing and at least one anchoring structure connected to the housing. The anchoring structure includes a micro-actuator, a micro-needle extending from the micro-actuator and a plurality of micro-darts extending from the micro-needle. The in vivo delivery device also includes a cap and a payload.

In accordance with some embodiments of the disclosed subject matter, provided is an anchor for an in vivo medical device deliverable within a patient's body. The anchor includes a micro-spring and at least one micro-needle having a plurality of micro-darts extending therefrom. The at least one micro-needle is connected to and forms a single, unitary, integral part with the micro-spring.

In accordance with some embodiments of the disclosed subject matter, provided is a method for delivering a payload within a patient's body. The method includes a step of providing an in vivo delivery device that includes a housing and at least one anchoring structure connected to the housing. The anchoring structure includes a micro-actuator, a micro-needle extending from the micro-actuator and a plurality of micro-darts extending from the micro-needle. The in vivo delivery device also includes a cap and a payload. The method includes steps of positioning the in vivo delivery device inside the body of the patient, allowing the in vivo delivery device to passively self-anchor to the tissue of the patient, and monitoring the patient.

### BRIEF DESCRIPTION OF THE DRAWINGS

The present disclosure will hereafter be described with reference to the accompanying drawings, wherein like reference numerals denote like elements.

FIGS. 1A-1D show a perspective view and certain close-up views of an anchoring structure;

FIGS. 2A-2D show a perspective view and a close-up view of an anchoring structure, as well as a demonstration of and force-graph of a compression cycle of an anchoring structure;

FIGS. 3A-3D show illustrations a device that includes an anchoring structure and a payload sensor, as well as placement of said device within a subject;

FIGS. 4A-4B show a device that includes an anchoring structure and an ingestible delivery system;

FIGS. 5A-5B show stabilization and covering options for a device that includes an anchoring structure;

FIGS. 6A-6B shows illustrations of a payload configured as an electrode array and an illustration of a particular electrode;

FIGS. 7A-7B show illustrations of a device having an anchoring structure and a payload configured as a therapeutic component;

FIGS. 8A-8D show an image of and illustrations of a device having an anchoring structure and a payload configured as a therapeutic component;

FIGS. 9A-9B show illustrations of a micro-needle having capillary channels;

FIGS. 10A-10D show images of a micro-needle having capillary channels;

FIGS. 11A-11C show images of a micro-needle having capillary channels for reagent loading and graphs of reagent loading/dispersion;

FIG. 12 is a flow chart depicting steps of various processes and methods that may be performed in accordance with the present disclosure;

FIG. 13A is a block diagram illustrating an example implementation of systems and methods according to the present disclosure;

FIG. 13B is a block diagram illustrating an example implementation of systems and methods according to the present disclosure.

### DETAILED DESCRIPTION

It will be appreciated by those skilled in the art that while the disclosed subject matter has been described above in connection with particular embodiments and examples, the present disclosure and the claims of the present disclosure are not necessarily so limited, and that numerous other embodiments, examples, uses, modifications and departures from the embodiments, examples and uses are intended to be encompassed by the claims attached hereto. The entire disclosure of each patent and publication cited herein is hereby incorporated by reference, as if each such patent or publication were individually incorporated by reference herein.

Provided is a device capable of fixing and/or anchoring certain payloads at given locations within the body of a subject, and methods of making and using the same. In certain embodiments, the device includes one or more micro-needles, one or more barbed-structures (which may also be interchangeably referred to herein as “micro-darts” and/or “micro-barbs”) extending from the one or more micro-needles, and one or more micro-actuators. In certain embodiments, the device can also include one or more payloads. In certain embodiments, one or more of the payloads can be a sensor. In certain embodiments, the sensor is a biosensor. In certain embodiments, the biosensor is configured for measuring neurotransmitters. In certain embodiments, the sensor/biosensor can include one or more microelectrodes. In certain embodiments, the micro-darts may be configured to facilitate improved tissue anchoring in a subject, for example along the wall of the human gastrointestinal (GI) tract. In certain embodiments, the one or more micro-actuators may include one or more actuating coiled structures (which may be interchangeably referred to as “micro-springs”) and/or one or more dissolvable caps. In certain embodiments, a micro-actuator may include one or more pre-compressed micro-springs and one or more caps formed from a dissolvable material (such as a dissolvable or meltable polymeric material). In certain embodiments, the one or more micro-actuators may be configured to apply a passive force. In certain embodiments, the one or more micro-needles may be formed on the end of the one or more micro-springs, such that the micro-spring may apply a force on the micro-needle, for example, to aid in tissue attachment. In certain embodiments, the payload (such as the micro-electrodes), micro-spring(s), micro-needle(s), and/or the micro-dart(s) may be formed using direct laser writing, 3D printing, and/or sputtering.

Provided is a tissue-attaching device which can be integrated into a variety of applications/tools for placement into the body of a subject (for example, into the GI wall of a human), for fixing and/or anchoring of certain payloads at given locations within the body of a subject.

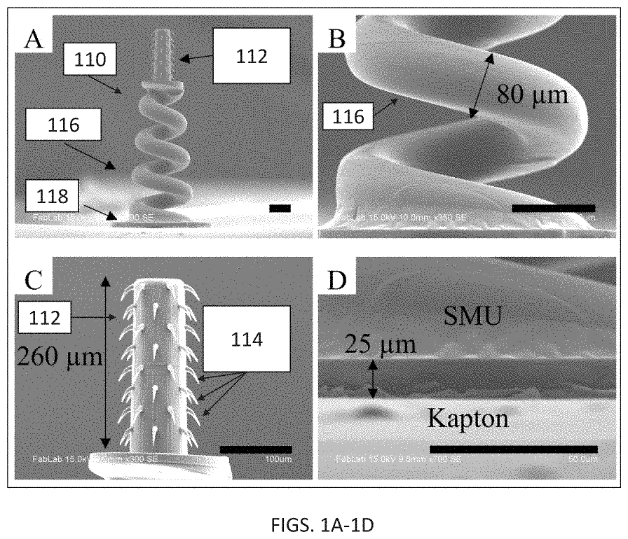

FIGS. 1A-1D show an anchoring portion 110 of a device 100. As shown in FIG. 1A, the anchoring portion 110 includes a micro-needle 112 having a plurality of micro-darts 114 (labelled in FIG. 1C) extending therefrom (which may be referred to collectively as one or more “barbed micro-needles”). The micro-needle is positioned on top of and extends from a micro-spring 116, which is a micro-actuator for the anchoring portion 110 of the device 100. The micro-spring 116 is connected to a base 118. A portion of the micro-spring 116 is shown in greater detail in FIG. 1B. The barbed micro-needle (including both the micro-needle 112 and the micro-darts 114) is shown in greater detail in FIG. 1C. In certain embodiments, the device 100 can also include one or more payloads, for example, one or more of the payloads can be a sensor.

Regarding the specific dimension of the embodiment device shown in FIGS. 1A-1D, FIG. 1A, shown is an anchoring structure 110 with a conical spring 116 (height; 660 μm) and barbed micro-needle 112 (height: 260 μm). FIG. 1B shows a zoomed-in view of the conical spring 116, showing the uniform diameter. FIG. 1C shows a barbed micro-needle 112 with 8 rows of sharp, backward facing micro-darts (also referred to as ‘barbs’) arranged in rows of 6 each. The base height of the micro-needle 112 is about 30 μm. FIG. 1D shows an intimate SMU/Kapton interface with base (height: 25 μm). The scale bars are 100 μm.

However, in some embodiments, an anchoring structure 110 may have other dimensions. For example, because some suitable processes may have a minimum feature size for components produced using DLW via TPP of approximately 100 nm and further because other manufacturing methods are traditionally used to produce components having a dimension in excess of about 10 mm, any component of an anchoring structure 110 may have one or more dimensions measuring from about 100 nm to about 10 mm if developed via a single process. For example, a micro-needle 110 may have a height from about 100 nm to about 10 mm and/or a width (i.e. cross-sectional diameter) from about 100 nm to about 10 mm. As another example, a micro-dart extending from a micro-needle may have a length from about 100 nm to about 10 mm and/or may have a minimum width (i.e. a minimum cross-sectional diameter) from about 100 nm to about 10 mm. As still another example, the wire forming a micro-spring may have a cross-sectional diameter of from about 100 nm to about 10 mm and/or a micro-spring may have a height from about 100 nm to about 10 mm. Moreover, any of the above-described dimensions for any of the above-described components may be from about 100 nm to about 1 mm, or from about 1 μm to about 1 mm, or from about 5 μm to about 1 mm, or from about 10 μm to about 1 mm, or from about 25 μm to about 1 mm, or from about 50 μm to about 1 mm, or from about 100 μm to about 1 mm, or from about 200 μm to about 1 mm, or from about 10 μm to about 800 mm, or from about 25 μm to about 700 mm, or from about 50 μm to about 600 mm, or from about 100 μm to about 500 mm, or from about 200 μm to about 400 mm.

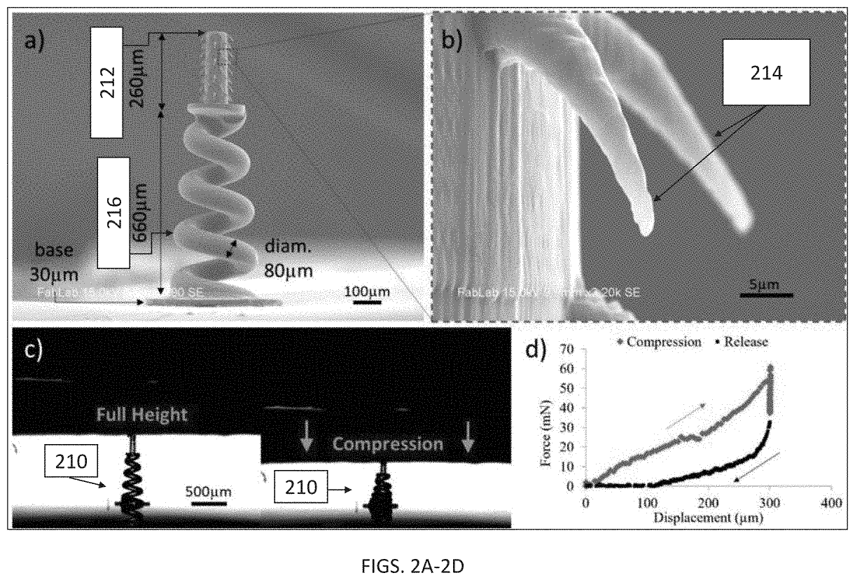

FIG. 2 shows additional details of the anchoring portion 110. FIG. 2A shows some potential sizes and scaling of the barbed micro-needle 112 and the micro-spring 116. FIG. 2B shows additional detail and potential size/scaling of the micro-darts 114. FIG. 1C shows the anchoring portion 110 in a compressed state and in a non-compressed (i.e., “full height”) state, while FIG. 2D shows a graph of the compression force (in mN) across the displacement of the anchoring portion 110.

More specifically, FIG. 2A shows an SEM image of a full anchoring structure 210, including micro-spring 216 and barbed micro-needle 212 (MN). Key dimensions are labeled. FIG. 2B shows a SEM zoomed on micro-barb structures 214. FIGS. 2C-2D show experimental testing of compression of anchoring structure 210. The images shown in FIG. 2C were taken with a high-speed camera (left) before and (right) after compression. FIG. 2D shows a Force vs. Displacement plot measured during compression and release of anchoring structure 210 (one representative cycle shown).

In one particular example, the tissue anchoring device may be positioned just past the epithelial barrier in the GI wall of a human subject. In such an example, the device may include a payload that is configured to measure submucosal 5-HT.

In certain embodiments, some or all of the device may be made using a method referred to as ‘direct laser writing’ (“DLW”) 3D printing via Two-Photon Polymerization (“TPP”). DLW via TPP enables complex designs with sub-micron resolution. In one example, DLW via TPP is used to construct micro-needles (MN) which include a biomimetic tissue-anchoring barbed micro-darts (MD), which can be referred to collectively as a “barded micro-needle”. In this example, certain aspects of the barbed micro-needle mimic certain structures found on some GI parasites. DLW via TPP can also be used to construct micro-spring. In one example, DLW via TPP can be used to construct a barbed micro-needle and a micro-spring that are integral to one another. TPP 3D printing enables facile design modifications, such as increasing the number of micro-electrodes, micro-needles, micro-darts, and/or micro-springs as needed. Additionally, using TPP 3D printing enables the footprint of the device to be scaled based on the number of features. In certain embodiments, the micro-spring can be configured for passive actuation and insertion of the barbed micro-needle into the tissue of a subject.

Utilizing DLW 3D printing via TPP processes to fabricate both microelectrodes and tissue-attaching micro-darts can facilitate the fabrication procedure of both elements, and provides the ability to modify designs as needed during the experimentation phase. The sub-micron x-y-z resolution and maximum print height of ˜1 mm can be utilized to design complex features with the high aspect ratio needed for microneedle design. As shown in FIG. 2A-2B, the biomimetic barb structures (i.e., micro-darts 216) are patterned around the exterior of the micro-needle 212, mimicking the tissue-attaching mechanism of the spiny-headed worm, a parasite which uses these barbs to invade the GI wall. This barbed micro-needle 212 can be fabricated on top of a micro-spring 216, wherein preloading via manual compression of the micro-spring 216 provides potential energy that can be released at the site of interest for passive actuation resulting in insertion of micro-needle 216 and attachment/anchoring of the device 200 within the tissue of a subject (for example, within human GI tissue). In some embodiments, the micro-actuator (such as micro-spring 216) may be held in a high-energy state, such as the compressed state of micro-spring 216, by a dissolvable cap. For example, the dissolvable cap could be represented by the compressive weight shown in FIG. 2C. In some embodiments, the dissolvable cap can be made of a temperature-sensitive polymer. In such an embodiment, the cap of temperature-sensitive polymer can hold the anchoring structure 210 (including the micro-spring 216) in its compressed state. In some embodiments, the dissolvable cap can be configured to melt at a particular temperature, such as at body temperature, to allow the micro-actuator (such as micro-spring 216) to release. In other embodiments, the anchoring structure 210 can be configured to include other actuation mechanisms. For example, the dissolvable cap can be altered to respond to a given environment, such as using pH or ion-responsive polymers.

In the embodiments shown in FIGS. 2A-2D, the anchoring structures 210 were designed using Autodesk inventor CAD software and exported for 3D printing and slicing using Describe software (Nanoscribe GmbH, Karlsruhe, Germany). However, in other embodiments, other suitable design and fabrication systems may be used. Specifically, the embodiments shown in FIGS. 2A-2D were produced using the following process: A pyrex substrate is cleaned, coated with a droplet of a near-infrared-curable resist, IP-S(Nanoscribe GmbH, Karlsruhe, Germany), and positioned upside down for Dip-in Laser Lithography (DiLL mode) printing. After printing, the structures are cleaned and UV cured until they are mechanically robust. A representative anchoring structure 210 is shown in FIG. 2A. The total height of the anchoring structure 210 is 950 μm, where the barbed micro-needle 212 is 260 μm in height and 74 μm in diameter, and the micro-spring 216 is 660 μm in height with an 80 μm wire diameter, standing atop a 30 μm base. The barbs (i.e., micro-darts 214) covering the micro-needle 212 (shown in FIG. 2B) are 8 μm at their base, decreasing to ˜1 μm resolution at the tip, and maintaining a downward curve dictated by the biomimetic design even when printed from bottom up. However, in other embodiments, other suitable materials and methods can be used to produce the anchoring structure 210, including the micro-needle 212, the micro-darts 214, the micro-spring 216, and the base 218.

In some embodiments, DLW via TPP may also be used to construct one or more payloads that can also be integrated into the device. For example, DLW via TPP can be used to construct micro-electrodes. In one example, said micro-electrodes may have tips that are selectively metalized by sputtering the whole surface, where doubly-reentrant structures prevent electrical connection between the tips and the rest of the cylindrical structure. Methods such as electrowetting and carbon nanotube (CNT) electrode modification can be utilized to improve binding and sensitivity of the micro-electrodes, for example for electrochemical 5-HT detection at these electrodes, although different modifying materials could be used to target different GI biomarkers (e.g., other neurotransmitters).

The specific anchoring structures 210 (including micro-springs 216) shown in FIG. 2C were able to be repeatedly compressed by 300 μm and completely recover to full height after release. The use of a conical spring (such as micro-spring 216) adds stability during compression, compared to a cylindrical spring. The force required for cycles of compression and release of the specific anchoring structures 210 (including micro-springs 216) were also assessed, and the results are shown in FIG. 2D). For anchoring structure 210, as the compressed displacement increased up to 300 μm, the force needed for compression increased. However, the force needed for compression remained stable at full compression of the anchoring structure 210. During release, the anchoring structuring 210 exerted an average force of average of 8 mM per 100 μm compression, which increased constructively in embodiments where more than one anchoring structure 210 was employed in an array. These results suggest that one or more anchoring structure(s) 210 can be configured to produce enough force to penetrate certain tissues of a subjection, for example the GI tissue of a human subject, by overcoming the 1.6 mN penetration force and 8 μN peristaltic force, and also that the anchoring structure 210 can resist removal via a high required pull-out force.

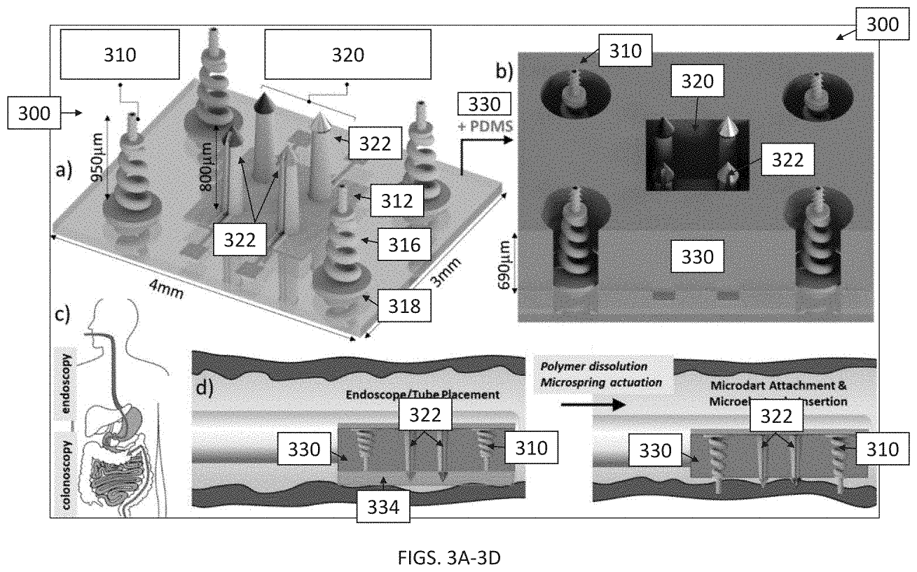

Some embodiment devices having one or more anchoring structures and one or more payloads may be placed within the body of a subject via endoscopic means (such as an endoscope). One example of placement of an embodiment device using endoscopic means can be seen in FIGS. 3A-3D. As shown in FIGS. 3A-3D, a polymer cap/coating 334 (shown in FIG. 3C) holds the anchoring structures 310 and the PDMS mold 330 in a compressed position. As shown in FIG. 3D, upon dissolution of the polymer cap/coating 334, the micro-springs 316 of the anchoring structures 310 release to actuate the anchoring structures 310 insertion of the barbed micro-needles 312 (including the micro-darts 314) into the GI wall. The tips of the micro-electrodes 322 are also inserted into the GI wall. The micro-electrodes 322 can then be used to measure submucosal 5-HT. Additionally, in certain embodiments, a device 300 can be positioned via an endoscope (e.g. colonoscope, nasogastric feeding tube, or other similar tool) and thereafter passively anchor to the tissue of the patient (via dissolution of the polymer cap/coating 334), while simultaneously remaining attached to the endoscope. Beneficially, some embodiments wherein a connection between a device 300 and an endoscope is maintained, can have wires to run electrochemical programs (or other functions) routed along the endoscope or other tool, as shown in in FIGS. 3C and 3D. For example, in some embodiments, wires run along the endoscope can connect a device 300 to an exterior computer, an exterior power source, or other device that is located outside of the patient. Some embodiment devices that maintain a connection between the device and the endoscope may be particularly well suited for delivery of sensors and/or short-term monitoring procedures.

In some embodiments, a device 300 having one or more anchoring structures 310 can be placed by an endoscope having a removable covering component. In some embodiments, the removable covering component of the endoscope may work in concert with the polymer cap/coating 334 of the device 300. In some embodiments, the use of an endoscope having a removable covering component may allow the polymer cap/coating 334 of the device 300 to be thinner and/or more reactive to the in vivo environment proximate the target tissue, thereby facilitating more rapid passive deployment of the anchoring structures 310. In some embodiments, the use of an endoscope having a removable covering component obviate the need for a polymer cap/coating 334, making the anchor an actively deployed rather than passively deployed embodiment. As such, in some embodiments, a device 300 may not include a polymer cap/coating 334.

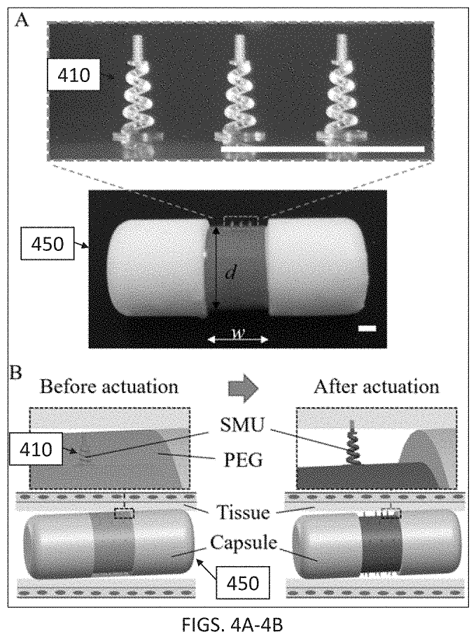

Additionally, there are further mechanisms for positioning anchoring onto specific tissues of a subject, other than placement via an endoscope tool (such as colonoscope, nasogastric, or a feeding tube). For example, as shown in FIGS. 4A and 4B, one or more anchoring structures 410 can be positioned on a surface of an ingestible delivery system 450, such as a capsule. In some embodiments, the ingestible delivery system 450 can contain a therapeutic agent, such as a drug. In some embodiments, the ingestible delivery system 450 can contain or include a power source or electrical conductor capable of administering therapeutic amounts of electricity to a localized portion of tissue (for example, for electroporation). In some embodiments, the ingestible delivery system 450 can contain or include a heat source for administering therapeutic heat to a localized portion of tissue (for example, for tissue ablation). In some embodiments, the ingestible delivery system 450 can be configured to deliver other therapeutic or monitoring technologies, such as dyes, reagents, sensors, scaffolding, or other materials or devices having desirable properties/functions.

In some embodiments, more than one anchoring structure 410 can be positioned on a surface of an ingestible delivery system 450. In the embodiment shown in FIGS. 4A and 4B, three anchoring structures 410 are be positioned on an outer surface of ingestible delivery system 450. However, it is contemplated that in other embodiments, other numbers of anchoring structures 410 can be positioned on a surface of ingestible delivery system 450. For example, in some embodiments, 5 anchoring structures 410 can be positioned on a surface of ingestible delivery system 450. In some embodiments, 10 anchoring structures 410 can be positioned on a surface of ingestible delivery system 450. In some embodiments, 20 anchoring structures 410 can be positioned on a surface of ingestible delivery system 450. In some embodiments, 30 anchoring structures 410 can be positioned on a surface of ingestible delivery system 450. In some embodiments, 50 anchoring structures 410 can be positioned on a surface of ingestible delivery system 450. In some embodiments, 100 anchoring structures 410 can be positioned on a surface of ingestible delivery system 450. In some embodiments, 500 anchoring structures 410 can be positioned on a surface of ingestible delivery system 450. In some embodiments, 1,000 anchoring structures 410 can be positioned on a surface of ingestible delivery system 450. In some embodiments, 1,000 anchoring structures 410 can be positioned on a surface of ingestible delivery system 450. In a particular embodiment, in light of the forces typically present in the human GI track, an ingestible delivery system 450 can have from 1 to 500 anchoring structures 410 positioned on a surface of ingestible delivery system 450.

In some embodiments, the anchoring structures 410 positioned on a surface of the ingestible delivery system 450 can initially be protected or covered, so that the anchoring structures 410 do not anchor the ingestible delivery system 450 prematurely and/or do not anchor the ingestible delivery system 450 to an inappropriate tissue of the subject. For example, the top portions (in particular, the barbed micro-needles) of the anchoring structures 410 positioned on a surface of the ingestible delivery system 450 can initially be protected or covered by a polymeric coating that prevents the anchoring structures 410 from contacting the tissue of a subject. In some embodiments, the protection or covering for the anchoring structures 410 can be removed. In some embodiments, the protection or covering for the anchoring structures 410 can be passively removable, for example, the protection or covering can be dissolvable and/or meltable. In some embodiments, the protection or covering for the anchoring structures 410 can be configured to be passively removable in the typical environment of the target tissue of a subject. For example, in some embodiments, the protection or covering for the anchoring structures 410 can be configured to be resolvable at the pH level that is typical of the environment surrounding the target tissue. In some embodiments, the protection or covering for the anchoring structures 410 can be configured to be meltable at a particular temperature that would typically be reached by the protection/covering at the time that the ingestible delivery system 450 reaches the target tissue. In some embodiments, when the protection or covering for the anchoring structures 410 is removed (e.g., is dissolved, melted, made sufficiently porous, etc.), the anchoring structures 410 are actuated, thereby coupling the ingestible delivery system 450 to the target tissue of a subject.

In some embodiments, the ingestible delivery system 450 to the target tissue can begin delivering the therapeutic/technology (i.e., the drug, the dye, the reagent, the sensor, etc.) to the target tissue of the subject simultaneously to the coupling of the ingestible delivery system 450 to the target tissue via the anchoring structures 410. In some embodiments, the ingestible delivery system 450 to the target tissue can begin delivering the therapeutic/technology (i.e., the drug, the dye, the reagent, the sensor, etc.) to the target tissue of the subject after the coupling of the ingestible delivery system 450 to the target tissue via the anchoring structures 410. In some embodiments, the ingestible delivery system 450 to the target tissue can continue delivering the therapeutic/technology (i.e., the drug, the dye, the reagent, the sensor, etc.) to the target tissue of the subject for a pre-determined duration, after the coupling of the ingestible delivery system 450 to the target tissue via the anchoring structures 410.

Referring again to FIGS. 4A and 4B, shown is a particular example of an ingestible tissue-anchoring apparatus having anchoring structures 410 configured as a 1×3 spring-microneedle unit (SMU) array wrapping around an ingestible delivery system 450 configured as a pill-sized dummy capsule (d: 9.5 mm, w: 5 mm, scale bars: 2 mm). Each SMU consists of a barbed MN on top of a conical micro-spring. The released SMU height (975 μm) is taller than the capsule trench (750 μm) so that the MN will be able to interact with the tissue when released.

Specifically, FIG. 4A shows an image of an ingestible delivery system 450 configured as a resident capsule integrated with a 1×3 SMU array compactly assembled on the surface trench (750 μm depth) of the capsule package using a flexible polyimide substrate (Kapton tape). Each micro-needle in each anchoring structure 410 is 260 μm tall with 48 barbs, allowing the robust tissue-anchoring performance demonstrated in the previous work. The backward facing barbs/micro-darts on the micro-needles enable the low tissue penetration force and simultaneous 10-fold higher pull-out force.

The conical micro-spring design allows reduced solid height for a compact design as each active coil fits within the next coil. In the particular example embodiment shown in FIG. 4A, the overall volume of the anchoring structure 410 is only about 5% of the overall capsule package volume. However, in other embodiments, anchoring structures of other sizes may be used. The conical design also provides lateral actuation stability as the base coils have larger diameters (250 μm to 100 μm) with less tendency to buckle than conventional compression springs. The design contains 4 spring coils with an 80-μm wire diameter, allowing stable directional actuation with an estimated spring constant of 340 N/mm and 100 mN peak compression force at 300 μm displacement. These calculations are based on the conical spring model, as well as the measured Young's modulus and shear strength of the IP-S photoresist. The design parameters and calculations of the conical micro-spring of the particular example embodiment shown in FIG. 4A, are listed in Table 1 below.

TABLE I
MICRO-SPRING DESIGN CHARACTERISTICS

| Symbol | Name | Value | |
|---|---|---|---|
| H | Height | 660 | μm |
| d | Wire diameter | 80 | μm |
| N | Number of coils | 4 | |
| D1 | Base coil diameter | 250 | μm |
| D2 | Top coil diameter | 100 | μm |
| E | Young's modulus | 4.6 | GPa [14] |
| τ [symbol] | Shear strength | >180 | MPa [15] |
| fₘₐₓ | Maximum spring displacement | 300 | μm |
| | or compression | | |
| F [symbol] | Peak compression force | 100 | mN |
| k | Spring stiffness | 340 | N/m |
| τ | Shear stress at fₘₐₓ | 158 | MPa |

[symbol]

indicates data missing or illegible when filed

In some applications, devices using anchoring structures positioned on a surface of an ingestible delivery system may be preferrable to devices requiring surgical or endoscopic placement, as

### Example 1—Biosensor (Gastrointestinal Serotonin)

In certain embodiments, the device can include a payload that is configured to be a sensor. In some embodiments, the sensor can be a biosensor. In some embodiments, the sensor can be configured to measure certain biomarker(s). In some embodiments, the sensor can include one or more micro-electrodes. In some embodiments, one or more of the micro-electrodes can be optimized for sensitivity to a certain biomarker(s). In some embodiments, the biomarkers can be a bio-signaling molecule, such as a neurotransmitter, for example serotonin.

Referring again to the embodiments shown in FIGS. 3A-3D and FIGS. 4A-4B, example embodiment device(s) including one or more anchoring structures as well as a payload are provided. In the example(s) shown, the payload is configured as a biosensor with one or more micro-electrodes in some application it may be useful to configure a device to measure serotonin. Specifically, FIGS. 3A-3D show certain structural and design aspects of such an embodiment, while FIGS. 4A-4B show certain aspects of insulating such an embodiment.

FIGS. 3A-3D show an embodiment device 300 including an anchoring structure 310 (that is substantially similar to anchoring structures 110 and 210, described above) and a payload 320 that is a biosensor 320. Further, the biosensor 320 is a micro-electrode array 320, with one or more micro-electrodes 322. The micro-electrodes 322 have been optimized to detect serotonin may be particularly beneficial because serotonin, or 5-hydroxytryptamine (5-HT), is an essential neurotransmitter and signaling molecule in the gastrointestinal (GI) tract. 95% of the 5-HT in the body is produced in the gut, mainly by enterochromaffin cells (ECCs) within the gut epithelium. ECCs release micromolar concentrations of 5-HT from their bottom, or basolateral, side where it can stimulate local enteric nerves to transmit sensations of nausea and pain and initiate peristalsis, contraction, and secretory reflexes. Altered regulation of 5-HT has been implicated in a wide variety of GI disorders, including inflammatory bowel disease (IBD), irritable bowel syndrome (IBS), autoimmune diseases such as celiac disease, and bacterial, viral, and parasitic infections. Indeed, GI disorders whose symptoms include nausea, diarrhea, abnormal GI transit, and visceral pain tend to correlate with increased clinical measures of luminal 5-HT and ECC count. 5-HT has also been implicated as a pro-inflammatory molecule in the GI tract, acting as an amplifier of immune responses and showing overexpression in animal models of GI inflammation, including bacterial infection and colitis.

Despite the clear clinical relevance of basolateral or submucosal 5-HT, no commercial technologies exist to measure this molecule in the relevant tissue in human patients. Current understanding of 5-HT levels in the gut comes from analyzing biopsy samples or luminal GI fluid via laborious and time-consuming methods (e.g., ELISA, HPLC). These methods do not lend themselves to real-time detection of 5-HT released from the basolateral side of the GI epithelium, where the release rate and concentration can dramatically impact nervous, muscular, and immune modulation. Electrochemical microelectrodes are capable of measuring 5-HT in the brains of anesthetized animals and in benchtop flow systems, but the electrodes must be handled and inserted manually. Arguably, the reason why these electrodes have not been used to study the neurotransmitter release in the gut is because of the difficulty maintaining stability in an organ which moves constantly, and also is only accessible through endoscopic means. Development of a microelectrode-based system capable of performing a sequence of in vivo measurements would more accurately capture 5-HT release patterns in response to applied stimuli, diet, or environmental triggers. Particularly, targeting basolateral 5-HT would provide a much more relevant picture of GI and enteric nervous system physiology. Furthermore, 5-HT is present at a higher concentration beneath the epithelium, since only a fraction of ECC-released 5-HT diffuses to the GI lumen, which may actually improve sensor performance compared to the luminal sensors which have been developed.

Referring again to the embodiment shown in FIGS. 3A-3D, the device 300 has a payload 320 that is a micro-electrode array 320 that is configured to measure gastrointestinal 5-HT levels in a human subject, wherein the device 300 can be positioned and anchored to the GI wall of the subject, proximate to the basolateral side of the enterochromaffin cells (ECCs) within the gut epithelium. The embodiment shown in FIGS. 3A-3D is a first demonstration of a modular passively-anchoring microelectrode system for measuring neurotransmitters in an in vivo GI tract, which would not depend on the use of complicated robotic actuation built into the endoscope or other tool. This embodiment could enable technologies which easily integrate into medical practice could improve the feasibility of doctors using GI 5-HT as a biomarker to grade the severity of their patients' disease, over the course of flare ups, treatment, and remission.

Specifically, FIGS. 3A-3D show a schematic of a device 300 configured as a GI 5-HT implant sensor. FIG. 3A shows a CAD diagram of system, indicating layout of anchoring structures 310 for tissue attachment surrounding a micro-electrode array 320 for submucosal 5-HT detection. Key dimensions are labeled. The micro-electrode array 320 includes four micro-electrodes 322 with contact pads: two Au-CNT working electrodes, one Au counter electrode, and one Ag/AgCl reference electrode.

FIG. 3B shows a diagram of the device 300 after a PDMS mold 330 has been added to the device 300, in order to stabilize structures (such as the anchoring structures 310 and the micro-electrodes 322) during compression and tissue insertion. FIG. 3B shows a cross section to reveal micro-springs 316 of the anchoring structures 310, within the PDMS mold 330.

FIGS. 3C and 3D show a diagram of GI tract access via endoscope and colonoscope (FIG. 3C), and diagrams of the implant process (FIG. 3D). In the embodiment shown in FIGS. 3C and 3D, the device 300 is attached to placement too (such as an endoscope, colonoscope, or nasogastric tube). The device 300 is then placed at the site of interest within a subject, for example near the GI wall. In certain embodiments, a dissolvable/meltable polymeric cap 334 can be used to keep the micro-spring(s) compressed until the device is placed at the site of interest.

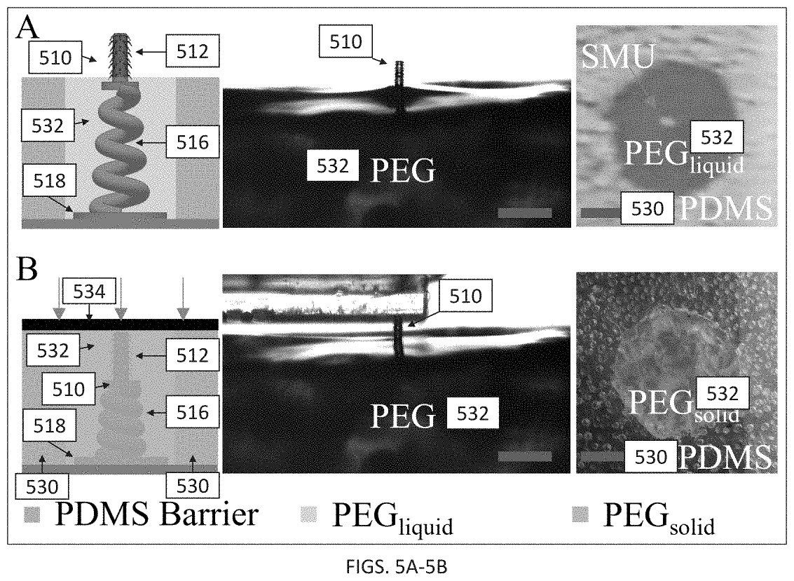

Moving now to FIGS. 5A and 5B show the stabilization and insulation of the device 400. For example, barbed micro-needle structures 410 can be stabilized and insulated via formation of PDMS molding 430.

More specifically, FIGS. 5A and 5B show one example assembly process for compressing an SMU (i.e., and anchoring structure 410) with a polymeric coating, in this case melted PEG. In this example, PEG was selected primarily because of its melting point (53-58° C.) and dissolution rate. However, in other embodiments other polymeric materials may be selected and/or other selection criterion may be evaluated. In this example embodiment, PEG was selected because the relatively low melting point allows the melted PEG at 100° C. to have long enough transition time before solidification for assembly. The particular example assembly process shown in FIGS. 5A-5B includes steps of: 1) preparing a 2-mm by 2-mm, 750-μm thick PDMS film and patterning the film with a 1-mm biopsy punch to create a central hole, 2) placing the PDMS well on the as-fabricated SMU substrate with the central hole and the SMU aligned concentrically, 3) adding a droplet of aqueous PEG which is pre-melted at 100° C. (FIG. 4A), and 4) pressing the tip of the SMN completely into the PDMS well and hold for 2 min so that the PEG will solidify and hold the compressed SMU in place (FIG. 4B). The SMU compression (225 μm) equals the height difference of the original SMU (975 mm) and the PDMS well (750 μm).

Referring again to the specific embodiment device 300 shown in FIGS. 3A-3D, the micro-electrode array 320 (of four micro-electrodes 322) is designed with a conical tip on a tapered cylinder, with a total height of 800 μm and half-max width of 140 μm. The micro-electrodes 322 include doubly-reentrant structures such that when a micro-electrode 322 is printed and sputtered with metal (e.g., Ti/Au), the conical tip is connected to the base via a trace down the side of the cylinder, but is not electrically connected to the rest of the cylinder, producing a specifically metalized tip.

In the example shown in FIGS. 3A-3D, the 3D printing process of these microelectrodes is similar to that describe with respect to the anchoring structures (110, 210, 310) above, but initial photolithography steps are taken first to ensure metal contacts are ultimately patterned correctly. In this example, the Omnicoat and SU-8 are spun onto a pyrex wafer, then patterned via UV exposure through a mask, revealing empty spaces for 3D printing the microelectrodes and their base, and routes from those electrodes to square contact pads. The wafer is then diced and micro-electrodes 322 are 3D printed, aligning the print with the SU-8 pattern. Then, the structures are sputtered with Ti and Au, then a paper mask can be applied over all but one microelectrode 322 which can be coated with Ag via Ebeam lithography. Omnicoat liftoff can then remove excess metal from the substrate. Au and Ag will remain on the entire 3D printed structures; however, in this example, the metals do not reach all the way into the reentrant structures around the tip circumference and down the length of the vertical trace. Therefore, these structures break the conductivity between the tip and the rest of the cylinder, facilitating use of the trace as a wire for external contact. Conductivity can be assessed using tools such as a micromanipulator probe attached to an ommeter.

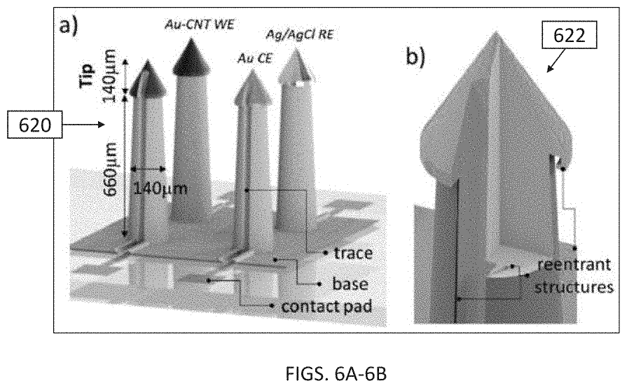

Referring now to FIGS. 6A and 6B, specific aspects of micro-electrodes 622 in micro-electrode array 620 are shown. FIG. 6A shows a CAD diagram of four-electrodes 622, including two Au-CNT WEs, one Au CE, and one Ag/AgCl RE. Key dimensions and features are labeled. FIG. 6B shows a close-up, cross-sectional view of one micro-electrode 622 (the Au micro-electrode). In FIG. 6B, the reentrant structures in the tip circumference of micro-electrode 622 (discussed above with respect to micro-electrodes 322, 522) are visible. The reentrant structures run along the vertical trace in the tip of micro-electrode 622, to separate tip from the rest of the cylinder structure.

The result from the fabrication steps, as described above for this example embodiment, is a microelectrode array with two Au working electrodes (WE), one Au counter electrode (CE), and one Ag reference electrode (RE). Electrodes can then be modified by electrowetting, in which electrodes are individually addressed by individually applying a voltage which wets the surface with an applied solution. For example, the working electrodes (WE) can be modified with a CNT solution in 1:1 ethanol and N-methyl-2-pyrrolidone. Electrowetting can also be used to selectively treat the Ag reference electrode (RE), for example, with FeCl3 to chemically convert it to Ag/AgCl, a standard RE material.

Beneficially, the example embodiment described above and shown in FIGS. 3A-3D, 5A-5B, and 6A-6B, provides integration of two novel high-resolution 3D printed structures, such that the embodiment can provide both (1) passive anchoring to the tissue of a subject and (2) in situ electrochemical molecular detection, which can be integrated onto a variety of tools already in use in the medical field. Some particularly beneficial aspects of the example embodiment are summarized below:
Biomimetic tissue-attaching mechanism, propelled by integrated micro-springs for passive actuation without manual handling or complex robotics.
Microelectrode printing and selective tip metallization using doubly-reentrant structures.
Individual electrode modification using electrowetting, such as Ag→Ag/Cl and Au→Au-CNT for stable and sensitive detection of 5-HT.
Application flexibility, resulting from potential modification of microelectrodes (such as through the use of different materials) to target detection of other relevant GI biomarkers, including other neurotransmitters or redox molecules.
Enhanced microelectrode electrochemical readings in the GI tract of living animals and humans resulting from the incorporated stabilizing structures, an improvement on manual handling of microelectrodes.
Protection of microelectrodes by the polymeric cap/coating, which keeps the anchoring structures compressed until localized at the site of interest. The polymeric cap/coating can be tuned to the needs of the GI area (e.g., pH, molecule or ion-responsive).
Small form factor (mm-scale) and modular design enables integration with many GI tools such as endoscopes, colonoscopes, or feeding tubes.

### Example 2—Drug Wafer/Pellet Delivery

Gastrointestinal (GI) disorders are typically managed by systemic administration of drugs (both oral and intravenous), resulting in broad dispersal of the therapeutic agents throughout the body. Many therapeutics for gastrointestinal disorders, like immune modulating agents and corticosteroids, are accompanied by adverse side effects that are a consequence of high levels of systemic drug absorption. When delivering these therapeutics systemically, excess agent is required to achieve adequate treatment at sites of interest.

Targeted treatment, achieved by delivery to specific locations in the GI tract, could offer comparable remediation of inflammatory sites without the use of excess therapeutic agent. Targeted delivery method serves to reduce side effects related to common GI drugs and lessen excess drug usage and, consequently, drug costs.

A variety of technologies currently exist to increase the regional specificity of drug delivery in the GI tract. Notably, pH-sensitive tablet coatings enable region-specific (stomach, small intestine, etc.) release of the encapsulated therapeutic agents. Additionally, mucoadhesive coatings can be leveraged to slow the transit of a tablet by attaching to mucus-lined tissue; thus, promoting focused release in a target region of the GI tract [2]. However, these technologies only enable broad regional targeting of drug delivery, making it impossible to direct treatment to specific locations in the GI tract. These technologies fail to address the need for location-specific delivery of therapeutic agents.

To address the need for a reliable mechanism capable of highly localized and sustained therapeutic delivery, certain example devices having biomimetic barbed microneedles are provided.

In some embodiment devices, the barbed microneedles can be attached to a therapeutic component, for localized deliver of the therapeutic. In some embodiments, the therapeutic component can be a solvent-cast water-soluble drug disk that distributes therapeutic agent through a diffusion process. Some embodiment devices can include a passively activated micro-actuator, for example a thermomechanical micro-spring actuator. In some embodiments, the therapeutic component and barbed micro-needles can be removed from the micro-actuator after anchoring to the GI mucosa.

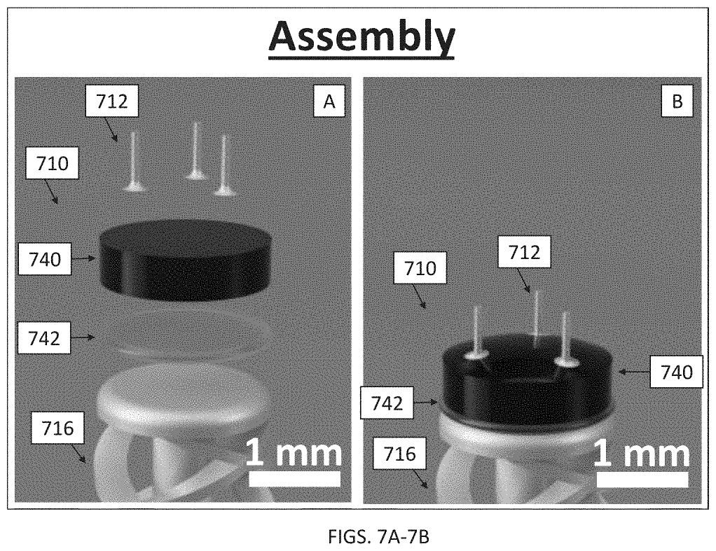

Referring to FIGS. 7A-7B, provided is an anchoring structure 710, having a plurality of barbed micro-needles 712, wherein each of the barbed micro-needles in the plurality of barbed micro-needles is positioned on top of a micro-spring 716. In the example shown in FIGS. 7A-7B, three barbed micro-needles 712 are disposed on the top of each micro-spring 716. However, in other embodiments, any suitable number of barbed micro-needles 712 can be disposed on the top of each micro-spring 716. For example, four barbed micro-needles 712 can be disposed on the top of each micro-spring 716, or alternative 6 barbed micro-needles 712 can be disposed on the top of each micro-spring 716, or 8 barbed micro-needles 712 can be disposed on the top of each micro-spring 716, or 12 barbed micro-needles 712 can be disposed on the top of each micro-spring 716, or 25 barbed micro-needles 712 can be disposed on the top of each micro-spring 716. However, in some embodiments, only 1 barbed micro-needles 712 may be disposed on the top of each micro-spring 716, or only 2 barbed micro-needles 712 may be disposed on the top of each micro-spring 716.

Specifically, FIG. 7A shows a fully assembled device 700 for anchoring a therapeutic to a tissue at a particular location within the body of a subject. The device 700 includes a plurality of anchoring structures 710, each of which includes a micro-needle 712 with a plurality of micro-darts 714 or barbs 714 extending therefrom (i.e., a “barbed micro-needle 712”). The device 700 also has a micro-spring 716, and a therapeutic component 740. In some embodiments, the device 700 can also include an adhesive component 742 positioned between the therapeutic component 740 and the micro-spring 718. The adhesive component 742 can couple at least one of the barb micro-needles 712 and/or the therapeutic component 740 to the micro-spring 716. In a particular example, the device 700 can be formed by attachment of the therapeutic component 740 to the micro-spring 716 via use of a PEG film (i.e., a particular adhesive component 742). In some embodiments, the barbed micro-needles 712 can then be adhered to the therapeutic component using an adhesive, such as Loctite M-21HP biocompatible (ISO-10993) medical device epoxy adhesive.

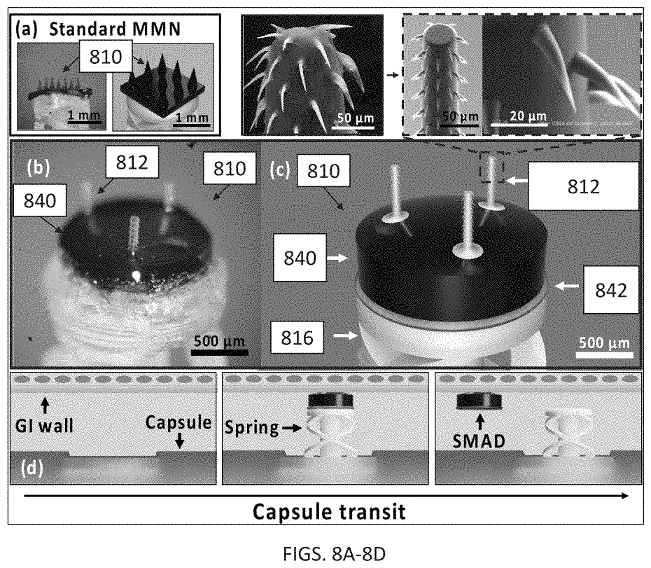

In the particular example embodiment shown in FIGS. 7A-7B and 8A-8D, the barbed microneedles 712 are coupled directly to a therapeutic component 740 which itself is couple to a micro-spring 716 via an adhesive component 742, to facilitate anchoring of the drug deposit (i.e., the therapeutic component 740) to target tissue within the body of a subject, and thereby enables long-term localized drug delivery. In this way, the example embodiment shown in FIGS. 7A-7B and 8A-8D addresses some or all of the above issues. For example, this example, this second example embodiment can facilitate treatment of lesions within the GI tract. This example, this second example embodiment demonstrates a 22-fold higher anchoring force than traditional conical molded microneedle (MMN) arrays, in ex-vivo intestinal tissue. Additionally, this example embodiment SMAD provides predictable drug delivery, locally, to a site of interest with comparable performance to MMNs, indicated by strong logarithmic correlation (R²=0.9773) across SMAD and MMN data.

Some embodiment devices, such as the particular example embodiment shown in FIGS. 7A-7B and 8A-8D, can be fabricated by a hybrid process that involves merging direct laser writing (DLW) of the barbed micro-needles 712 and solvent casting of model drug disks 640 (0=2 mm, t=500 μm) from 20% w/v polyvinyl alcohol (PVA) containing FD&C Blue #1, as shown in FIGS. 8A-8D.

Referring now to FIGS. 8A-7D, FIG. 8A shows an image and a rendering of a standard 3×3 conical molded microneedle (MMN) array attached to the thermomechanical spring actuator. FIG. 8B shows an image of a molded microneedle (MMN) array and a drug disk attached to spring actuator using water-soluble polyethylene glycol (PEG). FIG. 8C shows a schematic overview of a molded microneedle (MMN) array and a drug disk attached to spring actuator, which similar to the image shown in FIG. 8B. In the embodiments shown in FIGS. 8B and 8C, three barbed microneedles 812 are attached to the drug disk 840 using epoxy adhesive. FIG. 8D shows the actuation of a spring actuator 816, in an embodiment device where the spring actuator 816 is connected to the drug disk 840. The spring actuator 816 fires on command during transit through the body of the subject, for example, during transit through the small intestine of a human. In such an example, the molded microneedle (MMN) array and a drug disk is anchored in the mucosal tissue of the subject by the barbed micro-needles, leaving the deposit in the tissue to release the loaded drug by diffusion.

In the particular example embodiment shown in FIGS. 8A-8D, the barbed micro-needles 812 were fabricated using DLW, similar to the protocol described with respect to Example 1 above. The micro-needles 812 are 650 μm in height with a 74 μm tip diameter and a 300 μm flared base for enhanced adhesion to the drug disk. Each needle 812 contains a total of 72 backward-facing barbs 814 with high sharpness (˜1 μm) that promote robust tissue anchoring. DLW was performed using the Dip-in Laser Lithography (DiLL) mode on a fused silica substrate with the Nanoscribe Photonic Professional GT (Nanoscribe GmbH, Karlsruhe, Germany). Biocompatible (ISO-10993-5) IP-S photoresist was used with a 25× objective and a slicing distance of 1 μm to fabricate a 3-needle array. Needles 812 were printed upside down in a triangular pattern, supporting reliable attachment to the drug disk 840 and control over the spatial arrangement of the needles 812 on the fabricated structure. Post print, needle arrays were cleaned in propylene glycol monomethyl ether acetate (PGMEA) for 15 min, followed by 5 min in isopropyl alcohol.

In the particular example embodiment shown in FIGS. 8A-8D, the drug disks 840 were fabricated by solvent casting a film containing 20% w/v PVA (MW 31-50 kDa) (Sigma Aldrich, St. Louis, Mo., USA) with FD&C blue #1 dye to visualize drug diffusion. The solvent was allowed to evaporate for 24 h, then drug disks (0=2 mm) are punched from the resultant film. However, in other embodiments, the therapeutic component 840 may be formed by other suitable methods. Additionally, it is contemplated that other types of therapeutic components 840 could be used in certain embodiments, including some therapeutic components 840 having a semi-permanent housing (such as a capsule) couple to the micro-needles 812 and containing a therapeutic active and/or therapeutic components 840 have a permanent housing that is couple to the micro-needles 812 and through which a therapeutic active can diffuse (for example, through pores in the permanent housing). Alternative, it is contemplated that in some embodiments, the therapeutic components 840 could be a power source or electrical conductor capable of administering therapeutic electricity to a localized portion of tissue (for example, for electroporation). It is further contemplated that in some embodiments the therapeutic components 840 could be a heat source for administering therapeutic heat to a localized portion of tissue (for example, for tissue ablation.

In the particular example embodiment shown in FIGS. 8A-8D, the drug disks were first attached to the spring actuator 816 with ˜1.5 μg of melted polyethylene glycol (PEG). As can be seen in FIGS. 7A-7B, the disk is then lowered onto the flared bases of the 3-microneedle array and adhered using Loctite M-21HP biocompatible (ISO-10993) epoxy adhesive (Henkel Corporation, Stamford, Conn., USA). After curing the epoxy resin for 4 hours, the molded microneedle (MMN) array and a drug disk assembly is retracted, detaching the barbed microneedles from the fused silica substrate.

Additionally, molded microneedle (MMN) arrays were fabricated by solvent casting PVA containing FD&C blue #1 dye, an identical composition to the solution used for solvent casting of model drug disks. Polydimethylsiloxane (PDMS) microneedle molds were acquired from Blueacre Technology Ltd. (Dundalk, Co Louth, Ireland). The microneedle mold has a 11×11 array of 600 μm needles with a base diameter of 300 μm, and an interspacing of 600 μm on center. 500 μL of the PVA solution was deposited on the needle array mold, then placed under vacuum for 15 min to remove air from the needle mold voids. The solvent was allowed to evaporate for 24 hours, then a 3×3 needle array was cut from the molded part. The 3×3 array was then attached to the spring actuator using 1.5 μg of melted PEG.

Results—Mechanical tests were performed to compare SMAD and MMN tissue anchoring and removal forces. This was done using an Instron 5942 universal test apparatus (Instron Corporation, Norwood, Mass., USA) equipped with a 50 N load cell. All tests were performed using a crosshead speed of 1 mm/min. Spring actuators fitted with MMN or SMAD tip structures were lowered onto tissue samples until reaching the previously reported actuator force of 75 mN. Tissue samples were pre-coated with a ˜2 mm layer of 1×PBS (Sigma Aldrich, St. Louis, Mo., USA) to simulate the presence of mucus and aqueous intestinal media on the tissue surface. Upon reaching the 75 mN force, the tissue was moved 2 mm laterally to imitate the longitudinal motion experienced in the GI tract. The sample is then retracted, resulting in the detachment or sustained attachment of the respective tip structures. Detachment or removal force was measured for each sample to determine the strength of the tip structure attachment when compared to the anchoring force for each type of tip structure. Tip detachment force was determined as the removal force of the SMAD from the actuator.

For Model Drug Delivery testing, dye-loaded SMAD (n=5) and MMN (n=5) samples were applied to a thin agarose surface and dye diffusion was tracked at predetermined time intervals from 0 to 168 h. Images of each sample were captured at each time point in a light-controlled environment, enabling a quantitative image analysis approach for data interpretation using MATLAB R2021b (MathWorks Corporation, Natick, Mass., USA). From each image, the red color channel was isolated, and the resultant grayscale was binarized with a threshold intensity of 40%. The resulting binary matrix was used to determine the areal dye spread and diffusion radius as a function of time. After 168 hr, the agarose samples were submerged in DI water to allow the dye to be diluted to within the linear regime of optical density. Optical density of diluted samples was then obtained using a Molecular Devices SpectraMax Plus spectrophotometer (San Jose, Calif., USA). Optical density measurements were compared to a calibration curve to determine the initial dye mass for each sample. These values are used to account for concentration-related differences in diffusion behavior, enabling a more pertinent comparison of model drug delivery.

The SMAD was then evaluated qualitatively by lateral removal experiments to imitate the longitudinal motion of a capsule in the GI tract. Performance of the SMAD was then quantitatively compared to that of a 3×3 MMN array, looking at mechanical removal and anchoring properties as well as the dynamics of drug delivery from each structure.

Removal by Lateral Translation— A capsule in the GI tract will experience periodic peristaltic movements; thus, a significant component of force will be applied perpendicular to the actuation direction. To model this, the SMAD was attached to an actuator and translated laterally (FIG. 3) until the SMAD structure was removed. Removal occurs at approximately 3 mm deflection, corresponding to 3 mm of capsule transit within the intestine.

Firm tissue anchoring allows for removal of the SMAD from the actuator, but it also enables robust adherence to the target region and, consequently, reliable prolonged therapeutic delivery. In this respect, the term ‘Tip detachment force’ refers to removal force of the SMAD or MMN structure from atop the actuator, while ‘anchoring force’ refers to the force required to remove the SMAD or MMN structure from the tissue sample. The conical MMNs showed a low anchoring force of 0.8±0.1 mN compared to the 3.3±1.1 mN force required to detach the tip structure from the actuator. Conversely, the SMAD demonstrated an anchoring force of 17.2±2.6 mN, a 22-fold improvement over the conical MMNs and significantly higher than the detachment force. The exceptional anchoring ability of the structure compared to the MMNs affects more reliable tissue anchoring and system operation.

Also measured was the release and subsequent diffusion of dye from a SMAD sample at 0 hr, 48 hr, and 168 hr. At 48 hr, the visually discernable perimeter of dye diffusion is at a radial distance of ˜1.8 cm, while this expands to ˜2.5 cm after 168 hr. 5 samples of each SMAD and MMN were characterized using this diffusion approach.

Final squared diffusion radius (t=168 hr) shows a logarithmic correlation to initial dye mass (R2=0.9773) predicted by the solution to Fick's second law for radial diffusion distance. The logarithmic fit coefficient predicts a diffusion constant of D=2.6×10-10 m2/s that agrees strongly with previously reported value (D=(2.5±0.2)×10-10 m2/s) for dye diffusion in agar gel [15] confirming the relevance of the calculated logarithmic correlation coefficient.

Therefore, after correction for the initial dye mass in each sample, the SMAD and MMN data showed high correlation (R2=0.9773) indicating comparable performance. Overall, the robust anchoring provided by this system will enable location-specific and long-term anchoring of a drug deposit to facilitate prolonged treatment of target locations in the GI tract.

### Example 3— Capillary System Integrated Microneedles

Coated micro-needles provide a mechanically robust versatile delivery system capable of loading a broad spectrum of materials, ranging from small molecules to proteins, DNA, viruses, and microparticles into a subject. The efficacy of the coated micro-needles have been evaluated not only in transdermal delivery, but also for delivery via eye, vascular tissue, and the oral cavity. While various deliverables can potentially be coated on solid micro-needle surfaces, coating of the potential deliverables onto micro-needle surfaces with high uniformity and selectivity has been a challenging task for several reasons, including: 1) limited surface area for sufficient dosage, and 2) the need for both optimization in surface energy and viscosity of carrier liquids as well as the specifically designed instruments (e.g. screening masks, ink-jet printer) for controlled liquid introduction onto micro-needle.

The embodiments provided in this example solve some of these issues. The particular example embodiments leverage state-of-the-art 3-D direct laser writing (DLW) technology to realize system-level integration of the 3-D capillary components into micro-needles, enabling highly efficient and self-localizing therapeutics loading. These example complementary capillary system integrated microneedle (CCS-MN) enable autonomous localization of liquids carrying potential deliverables with a consistent 10 nL liquid loading per CCS-MN (compared to a 3 nL loading with conventional micro-needle design). Combined with the mechanical robustness for skin penetration, these example micro-needles demonstrate an innovation in the system-level design of next-generation micro-needle capable of achieving self-localized coating of potential therapeutic deliverables.

It is contemplated that the micro-needle (MN) and the complementary capillary system integrated microneedle (CCS-MN) structures disclosed in this example may be used with any of the anchoring structures and/or payloads described above. For example, it is contemplated that the MN and the CCS-MN structures disclosed in this example may be used in the design of micro-needles (112, 212, 312, 412, 512, 712, and/or 812) of anchoring structures (110, 210, 310, 410, 510, 710, and/or 810). Additionally or alternatively, it is also contemplated that the MN and the CCS-MN structures disclosed in this example may be used in the design of certain payloads, such as electrodes 322, 622 of electrode arrays 320, 620. Additionally, it is further contemplated that the MN and the CCS-MN structures disclosed in this example may be used with other payloads, to further improve the delivery of therapeutics and/or other reagents incorporated into said payloads. In certain embodiments, the MN and the CCS-MN structures disclosed in this example may be incorporated anchoring structures that are positioned on a surface of an ingestible delivery system. In some embodiments, the MN and the CCS-MN structures disclosed in this example may improve the anchoring of the anchoring structures and/or reduce the penetration force required for the anchoring structures to sufficiently couple with the target tissue. In some embodiments, the MN and the CCS-MN structures disclosed in this example may improve the efficiency of the payloads.

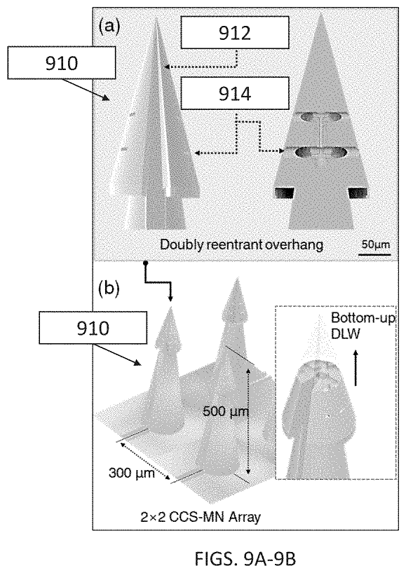

The specific example embodiments provided in FIGS. 9A-9B, 10A-10D, and 11A-11D show anchoring devices integrated with micro-needles having a complementary capillary system (CCS) that enables autonomous localization of therapeutic loading. Particular example micro-needles equipped with a complementary capillary system (a CCS-MN), can be realized by 3-D direct-laser-writing (DLW). Certain example CCS-MN can have a double-stacked cone structure with a unique top cone formed from opposing capillary actions. Certain example CC S-MN can have can be configured to include particular capillary structures/functions, such as external/internal capillary channels for rapid wicking/loading of liquid contents, respectively. Some example CCS-MN can have can be configured to include capillary structures/functions, such as a hydrophobic doubly-reentrant structure located at the bottom circumference for autonomous liquid confinement at the MN tip. A 2×2 CCS-MN array achieved robust skin penetration, withstanding 90 mN force while penetrating 300 μm into porcine skin. Beneficially, some example embodiment CC S-MN can provide significantly enhanced localization of liquid loading with the integrated CCS-MN when compared to conventional conical micro-needles, showing a 3.3-fold increase in loaded liquid volume (10 nL vs. 3 nL)—in good agreement with the qualitative staining test on the porcine skin from the delivered dye.

Now referring specifically to FIGS. 9A-9B, shown is a micro-needle 910 having an example complementary capillary system (CCS) including liquid-wicking capillary channels as well as the liquid-confining doubly reentrant overhang. The internal capillary channels 914 have a volume of ˜30 pL/MN. The internal capillary channels 914 were incorporated into the example CCS to address one of the major issues associated with coated micro-needles, the limited surface area, which makes it challenging to load sufficiently high therapeutic dosages. In the particular embodiment shown in FIGS. 9A-9B, the realization of the complex micro-needle structure was produced via DLW (Nanoscribe Photonic Professional GT), and IP-S proprietary resin was utilized for its relatively robust mechanical property (Young's modulus: 2.6 GPa). However, in other embodiments, other manufacturing methodologies (including other additive manufacturing methods) and other photocurable resins may be used. The embodiment CCS-MN shown in FIG. 9B displays 500 μm height (considering the epidermal depths (60-300 μm) for transdermal deliveries) with a spacing of 300 μm between the neighboring MNs in a 2×2 array. However, in other embodiments, other dimensions may also be used. For example, printing a larger array of the CCS-MN can be accomplished using DLW, if a longer printing duration is used (whereas the DLW of the 2×2 CCS-MN array lasted ˜20 mins with the Nanoscribe Professional GT).

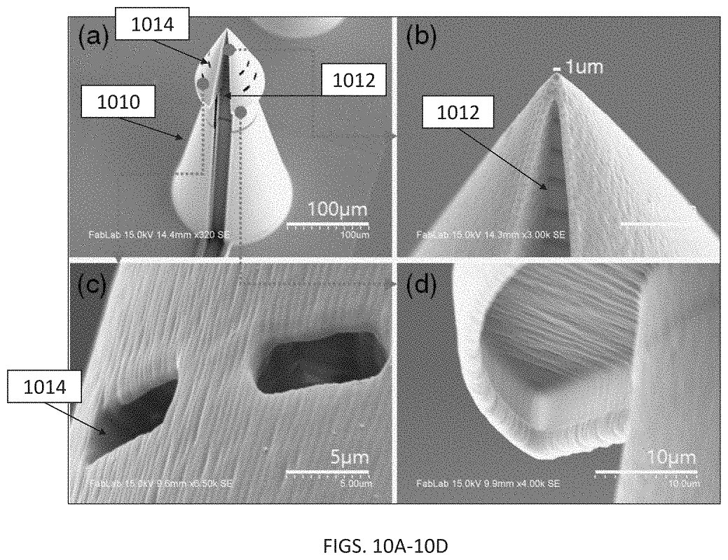

Referring to FIGS. 10A-10D, FIG. 10A shows an overview of the head of the micro-needle unit (the CCS-MN) showing the doubly-stacked cone structure with the CCS integrated at the top cone; FIG. 10B shows the 1 μm diameter MN-tip with the external capillary channel 1012 starting at the sharp tip for rapid liquid wicking; FIG. 10C shows inlets to the internal capillary channels 1014; and FIG. 10D shows the doubly-reentrant overhang at the circumference of the bottom of the top-cone, enabling self-localization of the introduced liquid.

The SEM images shown in FIGS. 10A-10D show the 3-D construction of the double-stacked cone shaped microneedle structure. The CCS components are integrated in the top cone of the micro-needle 1010, which displays excellent sharpness with the MN tip measuring 1 μm in diameter. This was achieved after optimizations of the key DLW parameters including the laser power and scan speed. Both the internal capillary channels 1014 and the external capillary channels 1012 were formed without any noticeable structural distortion. The development step after DLW, for removing excess photo-resin, facilitated the formation of hollow internal capillary channels 1014 while also maintaining adhesion between the printed structure and substrate (glass). The doubly reentrant structure located at the circumference of the top cone of micro-needle 1010, shown in FIG. 10D, closely resembles the doubly reentrant structure of previously described embodiments, ensuring structural hydrophobicity for autonomously confining liquid loading/coating at the top cone.

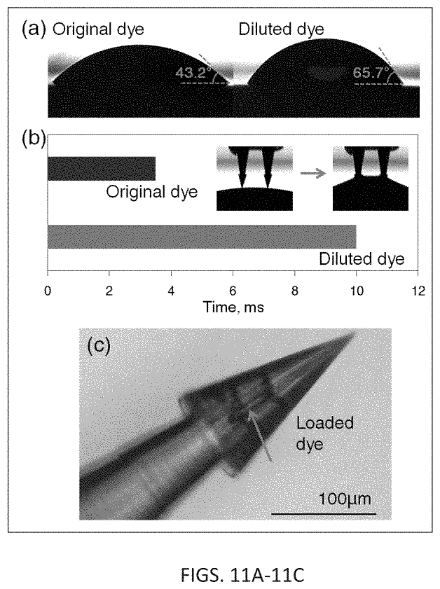

As shown in FIGS. 11A-11C, the polymeric CCS-MN showed a robust mechanical property for penetrating the porcine skin model, demonstrating robust insertion without any major disruptions/breakages as indicated by the linear change in force along the displacement, resisting up to 90 mN over 300 μm of penetration (effective penetration displacement).

In order to evaluate the enhanced liquid loading and delivery with the CCS-MN developed in this work, control MN samples displaying a conventional conical structure (i.e., single cone design with the absence of the top cone in CC S-MN) were utilized for comparison. The example embodiment CCS-MN demonstrated more dense skin coloration with the delivered dye contents compared to the conical MNs for both original and diluted dye cases, indicating the effective function of the embedded capillary channels for increasing the loaded liquid contents. Additionally, when the volume of the diluted dye loaded onto CC S-MN was analyzed using a calibration plot, the results indicated that the CCS MN carried ˜3.3 fold more of the dye solution compared to the conical MN (10 nL vs. 3 nL).

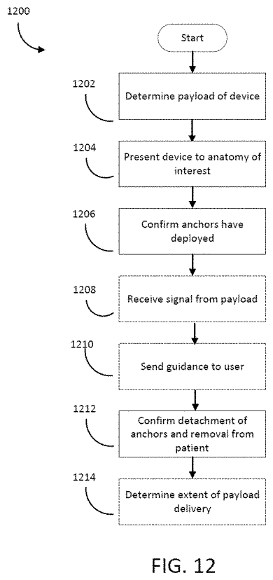

Referring now to FIG. 12, a flowchart 1200 is show which illustrates certain example steps of various processes that can be performed utilizing the techniques and components described above. The flowchart is not intended to be limiting of any method or process described herein, nor to define minimum steps required. Rather, the chart exemplifies certain steps which may be common to some embodiments, and the discussion below will also describe optional and additional steps which may be relevant to some embodiments.

In some embodiments, the flowchart 1200 may describe steps of a method for administering a medical anchoring device to a user, wherein the medical anchoring device has a payload. An initial step 1202 may be for a clinician or other user to determine the appropriate payload of the device. For example, it is contemplated that various devices could be manufactured so as to deliver different payloads. For some (such as sensors), these payloads may be determined at manufacturing time. For others, the payloads may be determined by the clinician, such as a therapeutic or drug being filled into a capsule or microneedle array, or a dissolvable drug disk being affixed to the device. In some embodiments, the type and dose of therapeutic may be selected by a clinician and loaded into the anchoring device or capsule at the time of administration. For example, in some embodiments, the device or capsule may be modular such that different dissolvable drug discs or liquid form of drug may be loaded into a portion of the capsule, then combined with the main assembly comprising actuatable anchors as described above.

At step 1204, the medical anchoring device with selected payload will be presented to the anatomy of interest. As discussed above, the particular anatomy of interest may vary, and in some embodiments may include various points along the GI tract, sinus tract, etc. For example, an anatomy of interest to which a clinician may want to anchor the medical device include a patient's trachea, stomach, small intestine, large intestine, sinus, other orifice, etc. A clinician may also select the medical device and/or the modality of deployment of the device depending upon the specific location to which the device should be attached. For example, for upper GI tract applications, it may be desirable to deploy the device via detachable connection to an endoscopy tube. In such embodiments, a clinician may insert the tube into a patient's GI tract and position the device at the anatomical location of interest while waiting for the anchors to actuate. In these embodiments, a clinician or other user can visually determine that the device has been presented to the correct anatomical location via optical feed from the endoscope camera. In other embodiments, such as where the anatomical location of interest may be in a patient's intestinal tract, a capsule delivery may be preferable. In those embodiments, a clinician may prescribe that a capsule containing an appropriate payload with a charged anchor mechanism be swallowed by a patient. The dissolvable cap of the capsule, in some embodiments, can be thickened or thinned so as to create a shorter or longer time to deployment of the capsule. In other embodiments, the material used for the dissolvable anchor cap can be modified so as to be dissolvable only in higher acidity environments, such as the interior of a patient's stomach. In this way, a method may include a step in which a clinician or other user selects a particular anchor actuating cap from among a variety of possible caps, to increase the likelihood a swallowed capsule will actuate its anchor system at the right region of a GI tract.

Next, at step 1206, a clinician or other user can confirm that the device's anchors have deployed and caused appropriate attachment. In embodiments involving deployment via an endoscope or other similar manual insertion, attachment can be determined visually through the optical feed of an endoscope. In embodiments involving a swallowed capsule, or where visual confirmation is not available, an external sensor can be used to detect positioning of the device. In these embodiments, the device may include internal circuitry to allow for ease of detection. As discussed above, the device may comprise passive location circuits, similar to RFID tags or other passive circuitry that can be utilized to detect the presence and location of the device. In other embodiments, active, signal-generating circuitry in the device may be utilized to detect the presence and location of the device. For example, the device may comprise a wireless communication transmitter or transceiver which can either periodically send signals which can be localized by a suitable sensor external to the patient, or can react to a signal sent by a detection device when the detection device is near enough to the device. In other examples, the location of the device could be determined magnetically via any suitable magnetic localization technique. Additionally or alternatively, the location of the device could be determined using a traditional endoscope, a capsule endoscope (such as a PillCam® device) and/or one or more sensors attached to an ingestible capsule. Moreover, the location of the device could be determined using external medical imaging devices (e.g. MRI, X-ray, Ultrasound). If the device is determined not to be moving from the anatomical location of interest (e.g., is not passing through the GI tract), then it can be inferred the anchors have deployed and made attachment.

At step 1208, in embodiments in which the payload of a device is optionally a sensor, the anchored device may be configured to generate signals indicative of the characteristics it senses within the body. In some embodiments, these signals may be transmitted via a wire connection (e.g., through an endoscopy tube) while in other embodiments these signals may be transmitted wirelessly. Thus, a technician or clinician utilizing the device may receive the signals from the device's payload and analyze them for clinical significance.

At step 1210, in some instances the signals received from an optional sensor of an anchored device may cause various messages to be presented to a user, e.g., comprising guidance regarding various actions or interventions that should be made. In some embodiments, these messages may indicate concerning levels of various biomarkers being detected. When a reading of a certain biomarker level (e.g., serotonin, etc.) is obtained by a sensor or reader (which, in some embodiments, may be a specialized belt, or a mobile device, etc.) the reading can be communicated via a communication network to a remote computer which can assess the reading and, based upon the biomarker level and patient characteristics, the computer can send a notice to a physician associated with the patient (e.g., via medical records applications or software, or via SMS message, etc.). In other embodiments, these messages may be meant for the patient, and may indicate that the patient should try to remain still, or refrain from eating, or other actions that may improve the payload's ability to acquire desired data.

At step 1212, the clinician or technician utilizing the device may confirm that the anchors of the medical device have detached from the anatomy of interest in one of several ways. In some embodiments, an endoscope or similar equipment may be utilized to dislodge the device from its anchored position. In other embodiments, the same detection equipment used to determine anatomical location of the device can be utilized to confirm the device naturally dislodged and passed the body.

At step 1214, in embodiments where the device was optionally configured to deliver a therapeutic or diagnostic agent (e.g., a drug, or dye), the device may be recovered after removal form the patient so that the clinician or technician can confirm the extent of the payload delivery. In other embodiments, the device may optionally have circuitry that emits a signal confirming full delivery of the payload.

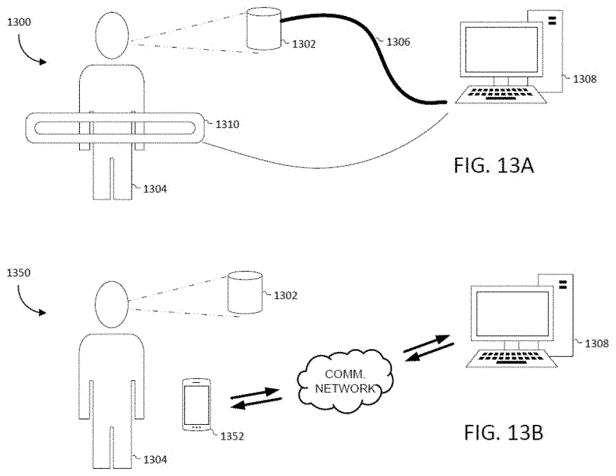

Referring now to FIG. 13A, a system diagram 1300 is shown, which depicts an example arrangement of medical devices, sensors, and computational devices for implementing the techniques and components described herein. As shown, a medical anchoring device 1302 has been prepared for administration to a patient 1304. In this embodiment, the medical anchoring device 1302 is configured (as described throughout this disclosure) for internal placement within the patient 1304 at an anatomical location of interest. Thus, the device 1302 is of a size and form factor that can facilitate placement within the patient 1304 by a physician (e.g., via an endoscopy tube 1306). In the embodiment shown, the device 1302 is detachably connected to the end of an endoscopy tube 1306, such that a clinician can release and deploy the device within the GI tract of patient 1304. In such embodiments, the device 1302 may comprise its own internal power source, such as a small battery, as well as wireless communication circuitry similar to that of a PillCam® device. Thus, the clinician can visualize placement of the device 1302 via the native camera of the tube 1306 through a computer 1308 connected to the tube 1306. The device 1302 can be detachably connected to tube 1306 in several ways, including a dissolvable adhesive, mechanical or magnetic connection, or the like. For example, a component or adhesive connecting the device 1302 to the tube 1306 can be configured to dissolve in the in vivo environment typically found proximate the target tissue. For example, a component or adhesive connecting the device 1302 to the tube 1306 can be configured to melt and/or deactivate at the temperature typically found proximate the target tissue. As another example, a component or adhesive connecting the device 1302 to the tube 1306 can be configured to dissolve and/or deactivate at the pH typically found proximate the target tissue. As still another example, a component or adhesive connecting the device 1302 to the tube 1306 can be configured to break or fail upon exposure (of sufficient duration) to the forces typically found proximate the target tissue. In some embodiments, at least a portion of one or more of the components of device 1302 (e.g. an anchoring structure, a micro-needle, a micro-spring, etc.) can be configured to dissolve, melt, or otherwise breakdown after sufficient exposure to the environment typically found proximate the target tissue. In a particular embodiment, the entire device 1302 can be configured to dissolve, melt, or otherwise breakdown after sufficient exposure to the environment typically found proximate the target tissue.

Once the device 1302 is anchored at the location of interest within patient 1304, it may be desirable to monitor output signals of sensors (such as electrodes) of the device 1302. For example, as described in this disclosure, the device 1302 may comprise electrodes configured for detection of various biomarker levels depending upon where in the body the device 1302 is located. While examples of electrodes are described above as potential payloads for device 1302, other types of sensors are contemplated as the payload. For example, levels of acidity could be measured (e.g., by including dissolvable dielectrics or utilizing electrodes whose conductivity changes by PH level, etc.), temperature could be measured through known means, ECG measurements can be taken, etc. As another example, levels of tissue impedance from one electrode to another electrode could be measured through known means, and/or measurement of tissue impedance from the tip of a needle to the base of a needle. Beneficially, tissue impedance measurements could provide insight into tissue integrity and fluid content of the tissue. Additionally, the presence of and/or the concentration of certain biomarkers (such as glucose, DNA, and particular antibodies) could be sensed using suitable functionalization of embodiment electrochemical sensors. For transmission or certain information, 495 MHz medical communication frequency could be used, as well as Bluetooth 2.45 GHz, or inductively using RFID. In some embodiments a signal monitoring device 1310 may be utilized to receive signals transmitted from the device 1302 indicative of the measurements taken by the sensors of the device's payload. Thus, in some embodiments, the device 1310 may comprise a transmitter for sending signals to receivers external of the patient's body 1304. As shown the monitoring device 1310 may include one or more receivers, which may be implemented in a band that can be worn about the patient's torso or other part of the patient's body. The monitoring device 1310 can be connected to a computer 1308 which may comprise software causing the computer to process and interpret the signals detected by monitoring device 1310, e.g., within the same exam room or clinic or other facility that is treating the patient. In other embodiments, the monitoring device may itself include a processor and memory for storing and interpreting the signals received from device 1302.

Referring now to FIG. 13B, an alternative arrangement 1350 is shown for deploying a medical device and performing some of the methods described herein. In the embodiment of FIG. 13B, a medical anchoring device 1302 may be configured as a capsule or other swallow-able form factor. Where the device 1302 is to be swallowed, the size and contour of the device 1302 may be similar to that of a standard pill, such as approximately 10-12 mm by 25-27 mm, or other similar sizes. Similarly, where the device is to be introduced to the patient's body 1304 through another orifice, the size and shape of the device can be suitably modified. As described above, the device 1302 may contain anchors at a distal end of the capsule, or in a middle portion of the capsule, or at other desirable locations.

Once the device 1302 is introduced to the anatomy of interest of the patient 1304 and anchored, the device may transmit signals indicative of measurements it is taking. For example, the device may emit a signal indicative of the levels of administration of a therapeutic payload of the device (e.g., as a drug disc dissolves, an electrical attribute of a sensor may change (e.g., a capacitance or the like), or may emit signals indicative of a physiological attribute or biomarker to be measured by the device 1302. In one embodiment, these signals may be detected by a patient's mobile device 1352, wearable device, or accessory attachable to the mobile device. The patient's own mobile device 1352 may, thus, comprise software that causes the device to record and store data obtained from the anchored medical device 1302, or may comprise software that operates a separate sensor accessor to obtain the data. The mobile device 1352 may then transmit the data via a communications network to a computer 1308 associated with the patient's physician and/or medical record.

Various features and advantages of the various aspects presented in the present disclosure are set forth in the following claims.

What is claimed is:

1. An in vivo delivery device for attaching to tissue within the body of a patient, the in vivo delivery device comprising:
a housing;
at least one anchoring structure having:
a micro-actuator;
a micro-needle extending from the micro-actuator; and
a plurality of micro-darts extending from the micro-needle;
a cap; and
a payload.

2. The in vivo delivery device of claim 1, wherein the micro-actuator is a micro-spring.

3. The in vivo delivery device of claim 2, wherein the micro-spring is a conical micro-spring.

4. The in vivo delivery device of claim 3, wherein the at least one anchoring structure is formed via direct laser writing 3D printing via two-photon polymerization.

5. The in vivo delivery device of claim 1, wherein the payload comprises a sensor.

6. The in vivo delivery device of claim 5, wherein the sensor comprises a plurality of electrodes.

7. The in vivo delivery device of claim 6, wherein the plurality of electrodes are at least partially surrounded by polyethylene glycol (PEG).

8. The in vivo delivery device of claim 6, wherein the plurality of electrodes comprises:
at least one electrode having a gold (Au) coating;
at least one electrode having a silver (Ag) coating;
at least one other electrode; and
wherein the electrodes are configured to detect changes in the concentration of a biomarker.

9. The in vivo delivery device of claim 8, wherein the biomarker is serotonin.

10. The in vivo delivery device of claim 1, wherein the payload is a therapeutic.

11. The in vivo delivery device of claim 10, wherein the therapeutic is a drug disk.

12. The in vivo delivery device of claim 1, wherein the payload comprises one or more micro-needles having a plurality of interconnected internal capillary channels.

13. The in vivo delivery device of claim 12, wherein the payload comprises one or more micro-needles having at least one external capillary channel.

14. The in vivo delivery device of claim 13, wherein the payload is configured to deliver a fluid into the tissue of the subject via one or more of the capillary channels.

15. The in vivo delivery device of claim 1, wherein the patient is a human, and
wherein the device has a total size and a profile suitable for delivery into the body of the patient via the patient's gastrointestinal (GI) tract.

16. An anchor for an in vivo medical device deliverable within a patient's body, the anchor comprising:
a micro-spring;
at least one micro-needle having a plurality of micro-darts extending therefrom, wherein the at least one micro-needle is connected to a distal end of the micro-spring; and
a dissolvable cap formed over the micro-spring and micro-needle, positioned such that the micro-spring is releasably held in a compressed position by the dissolvable cap.

17. The anchor of claim 16, wherein the at least one micro-needle comprises a plurality of micro-needles, and wherein the micro-spring is connected to and forms a single, unitary, integral part with at least two micro-needles.

18. The anchor of claim 16, wherein the micro-spring is connected to and forms a single, unitary, integral part with a single micro-needle.

19. The anchor of claim 16 further comprising a payload,
wherein the at least one micro-needle comprises a plurality of micro-needles,
wherein the payload is connected to at least three micro-needles, and
wherein the payload is connected to the micro-spring.

20. A method for delivering a payload within a patient's body, comprising:
providing an in vivo delivery device comprising:
a housing;
at least one anchoring structure having:
a micro-actuator;
a micro-needle extending from the micro-actuator; and
a plurality of micro-darts extending from the micro-needle;
a cap; and
a payload;
positioning the in vivo delivery device inside the body of the patient;
allowing the in vivo delivery device to passively self-anchor to an anatomy of interest of the patient; and
monitoring the in vivo delivery device until removal from the patient's body.
